(19) United States
(12) Patent Application Publication
(10) Pub. No.: US 2018/0185526 A1
(43) Pub. Date: Jul. 5, 2018

(54) Apparatus and Method for Using Ultraviolet Light with Pulsatile Lavage

(71) Applicant: Kevin Brown, Winterville, NC (US)

(72) Inventor: Kevin Brown, Winterville, NC (US)

(21) Appl. No.: 15/909,551

(22) Filed: Mar. 1, 2018

Related U.S. Application Data
(63) Continuation of application No. 13/960,589, filed on Aug. 6, 2013.
(60) Provisional application No. 61/680,061, filed on Aug. 6, 2012.
(60) Provisional application No. 61/751,490, filed on Jan. 11, 2013.

(51) Int. Cl. A61L 2/00

(52) U.S. Cl. CPC A61L 2/0047; A61L 2202/21

(57) ABSTRACT

An illumination device comprises a housing, a power supply and at least one light source powered by the power supply. The at least one light source is adapted to emit radiation from the housing at one or more therapeutic wavelengths. The illumination device is positioned in proximity to an area for applying radiation to the area for a time and intensity sufficient to have a bacteriocidal effect.

### RELATED APPLICATION

The present application is a continuation of U.S. application Ser. No. 13/960,589 filed Aug. 6, 2013, which claims the benefit of U.S. provisional application No. 61/680,061, filed Aug. 6, 2012, and U.S. provisional application No. 61/751,490, filed Jan. 11, 2013. These applications are each hereby incorporated by reference in their entireties.

### BACKGROUND

An apparatus and method for using ultraviolet light (UV) with pulsatile lavage is described and, more particularly, an apparatus and method using UV light with a pulsatile lavage device at a surgical or wound site.

Surgical site infections occur after an invasive procedure at the site of the surgery. Every year in the U.S. there are nearly 1.7 million occurrences of surgical site infections with approximately 99,000 cases resulting in death. Surgical site infections are an obvious burden to patients and physicians, but also to hospitals, requiring billions in treatment costs.

A current approach to reducing surgical site infections is pulsatile lavage. A pulsatile lavage device is a manual irrigation device that directs a pressurized stream of saline fluid directly at a wound in order to disinfect and debride the wound. The pulsatile lavage device injects fluid into a wound area and uses a suctioning mechanism to remove debris and necrotic tissue.

Ultraviolet light of a particular range of wavelengths, intensities, and durations can kill or inhibit growth of microorganisms. Specifically, ultraviolet radiation in the range of 200 nanometer (nm) to 300 nm is effective against airborne and surface bacteria, viruses, yeasts, and molds. For most microorganisms, the peak inactivation wavelength is at or about 260 nm. Mercury lamps produce UV light very efficiently at 254 nm and, therefore, this wavelength has become a standard wavelength.

There is a need for an apparatus that combines UV light with pulsatile lavage for use at a wound or surgical site. The combined device will bring the germicidal capabilities of UV light to conventional pulsatile lavage in order to further decontaminate wounds and reduce surgical site infections.

### SUMMARY

An illumination device is described and comprises a housing, a power supply and at least one light source powered by the power supply. The at least one light source is adapted to emit radiation from the housing at one or more therapeutic wavelengths. The illumination device is positioned in proximity to an area for applying radiation to the area for a time and intensity sufficient to have a bacteriocidal effect. In one aspect, the radiation emitted is ultraviolet C radiation for a duration of between about 5 seconds and 2 minutes.

A further illumination device is described and comprises a housing, a power supply and at least one light source powered by the power supply. The housing is configured to be mounted to a pulsatile lavage device. The at least one light source is adapted to emit radiation from the housing at one or more therapeutic wavelengths. The illumination device is positioned in proximity to an area for applying radiation to the area for a time and intensity sufficient to have a bacteriocidal effect.

### BRIEF DESCRIPTION OF THE DRAWINGS

FIG. 1 is a front side perspective view of an embodiment of an apparatus for using ultraviolet light with a lavage device.

FIG. 2 is an exploded perspective view of an apparatus for using ultraviolet light with a lavage device as shown in FIG. 1.

FIG. 3 is a top plan of an ultraviolet light unit for use with the apparatus for using ultraviolet light with a lavage device as shown in FIG. 1.

FIG. 4 is a longitudinal cross-section of the ultraviolet light unit as shown in FIG. 3.

FIG. 5 is a front side perspective view of another embodiment of an apparatus for using ultraviolet light with a lavage device.

FIG. 6 is a circuit diagram for use with an apparatus for generating ultraviolet light.

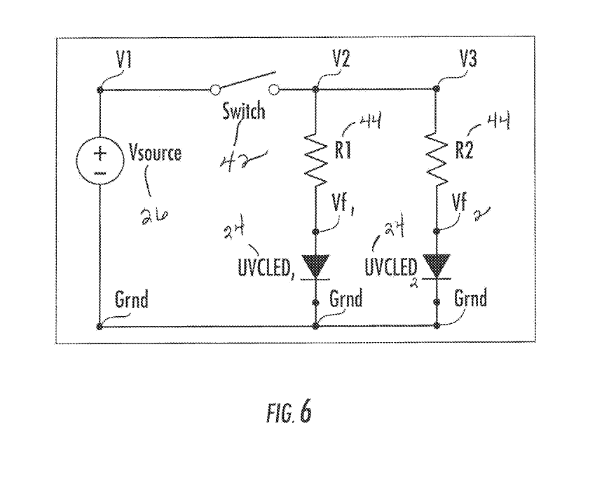

### DETAILED DESCRIPTION

Certain terminology is used herein for convenience only and is not to be taken as a limitation on the invention. For example, words such as “upper,” “lower,” “left,” “right,” “horizontal,” “vertical,” “upward,” and “downward” merely describe the configuration shown in the FIGs. Indeed, the components may be oriented in any direction and the terminology, therefore, should be understood as encompassing such variations unless specified otherwise.

It is understood that although a UV light apparatus is described in detail herein with reference to an exemplary embodiment for use with pulsatile lavage, the UV light apparatus may be applied to, and find utility in, other devices and tools for medical and non-medical use. As described above, UV light is used in a wide variety of applications for disinfecting or sanitizing areas of exposure. Therefore, although the UV light apparatus will be described in detail herein as embodied in a device for medical use, it is not intended to be so limited. Moreover, the UV light apparatus may be used as a stand alone device and not necessarily in combination with any other device or function. Thus, the UV light apparatus described herein has general applicability to any circumstance wherein improvements in disinfection and sanitization are desired.

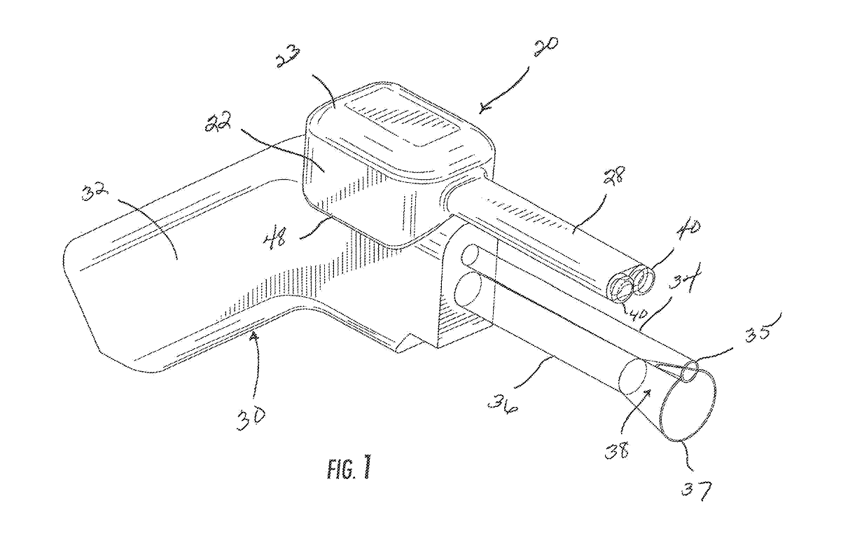

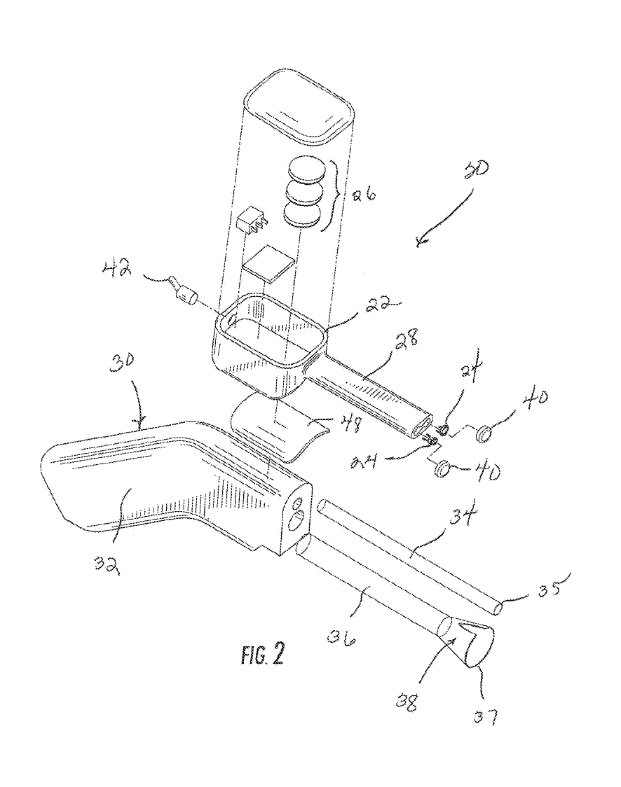

Referring now to the drawings, wherein like reference numerals designate corresponding or similar elements throughout the several views, an embodiment of an apparatus for using ultraviolet light with a pulsatile lavage device is shown in FIGS. 1 and 2 and generally designated at 20. The UV light apparatus 20 comprises a housing 22 configured to be mounted to the lavage device 30. The housing 22 accommodates a UV light apparatus, including a UV light source 24 and a power source 26 to power the UV light source for delivering UV light to a patient. A tubular wand 28 extends from the housing 22 to a distal outlet through which the UV light is emitted.

The housing 22 is substantially cube-shaped, although the housing may be formed in various other suitable shapes and sizes. The housing 22 may be formed of a relatively rigid material to protect the components. In one embodiment, the housing is constructed from a rigid plastic, such as polyvinyl chloride. A removable cover 23 is provided for selective access to the interior of the housing 22, such as for removing or replacing batteries when used as the power source 26.

The UV light source 24 is disposed within the housing 22 and is adapted to emit UV light to an area being treated. In one embodiment, the light source 24 comprises a light emitting diode (LED). A suitable LED for this application is available from HexaTech. In the FIGs., two LED's are shown for producing a desired optical intensity. The LED's 24 are positioned at the outlet of the tubular wand 28 projecting from the housing 22. This arrangement improves light localization at the wound site, minimizes scatter and enhances the germicidal effect. In addition, selecting the length of the wand 28 allows for adjustment of the UV dosage. The outlets of the wand are covered by lenses 40 to protect the LED's 24. Intermediate wires or cables (not shown) operatively connect the LED's 24 and the power source 26 for delivering power to the light source.

In another embodiment, the light source 24 may include a mercury-vapor lamp, such as a mercury vapor low pressure lamp or a medium pressure lamp. Other embodiments of the UV light apparatus 20 may comprise a plurality of UV light sources, which may be the same or may be different. It is understood that the UV light source 24 may be remote from the housing 22, wherein an optical cable (not shown) is operatively connected to the light source 24 for delivering UV light to the desired area.

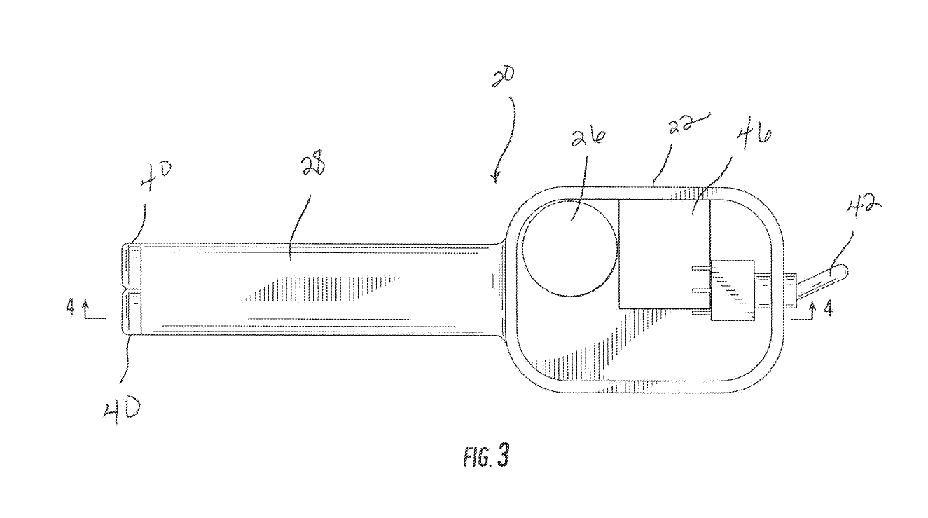

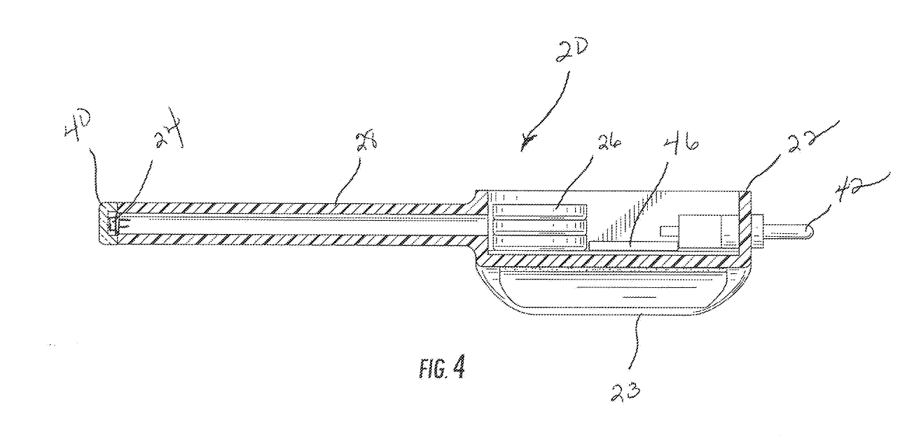

The power source 26 may include one or more disposable or rechargeable batteries, which fit within the housing. In one embodiment, the batteries are alkaline or lithium batteries. For example, three coin cell batteries are shown in the FIGS. 3 and 4. When combined, the three 3V batteries produce 9V of power for powering the LED's 24. A beryllium metal strip (not shown) may be placed at the top and bottom, respectively, of the stack of batteries, providing a conductive surface with a large contact area to which the power wires or cables can be soldered. Dielectric tape can also be used to hold the battery stack together and insulate the batteries from other metal parts of the circuit.

Alternatively, the power source 26 may be remote from the light source 24 with a power cord extending between the power source and the light source. An external power source 24 can also be used, such as an AC power supply connected to the light source 24 via one or more wires or cables.

A switch 42, shown as a toggle switch (FIGS. 2-4), is provided to actuate the power source 26 for turning the light source 24 on and off. The switch 42 may be positioned at any preferred location on the housing 22. Electrical connections (not shown) extend between the switch 42 and the power source 26 within the housing 22.

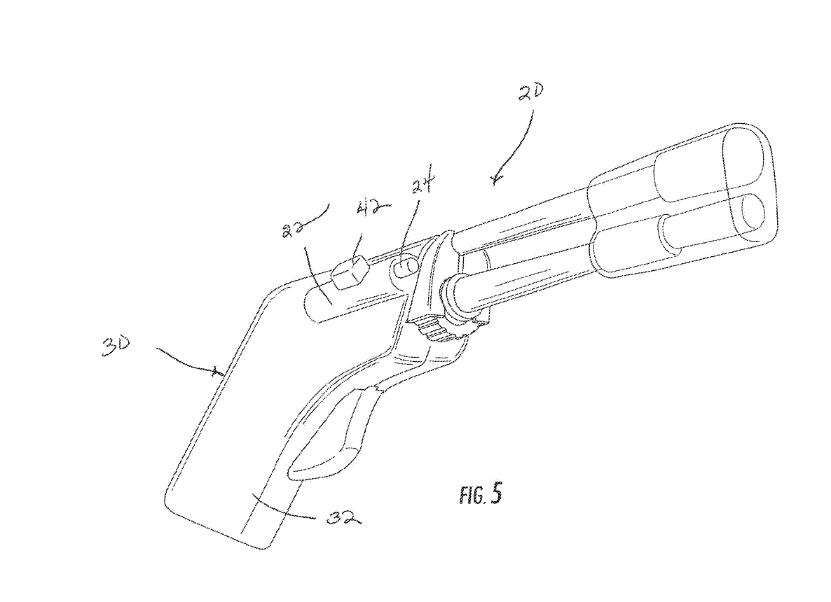

An embodiment of a circuit for the UV light apparatus 20 is shown in FIG. 5. The circuit comprises the power source 26, the switch 42, two sets of resistors 44 and two LED's 24. Each resistor is wired in series with an LED and the LED's are wired in parallel with each other. The components of the circuit are soldered onto a circuit board 46 (FIGS. 3 and 4). As described above, the switch 42 is connected to the power source 26 and the first branch of the circuit, including the first LED (UVCLED1). When the switch 42 is closed, the circuit is complete and current can flow to the LED's. In the closed circuit, V1=V2=V3. The intensity of each diode is determined by the amount of forward current that flows through the diode. Therefore, the intensity of the diode will be a function of the associated resistor 44 and the power source 26. LED's have a maximum forward current to which they can be exposed before failure. To avoid failure of an LED, the resistance values are chosen such that the forward current will be high enough to produce sufficient optical intensity while not exceeding the maximum forward current specification for the LED. The Node Voltage Method is used to determine the proper resistance values. For example, the Node Voltage Equation for branch 1 (UVCLED1) is:

V₂−V^{f1}/R₁=I_{f1}

where If1 is the maximum forward current for the LED. The same analysis seen above can be applied to each branch of the circuit depending on the type of LED used. The configuration of the circuit will remain the same, but the resistors and power source can change to accommodate different LED's.

The lavage device 30 comprises a body 32 for accommodating an irrigation system and a suction system. In one embodiment, the body 32 of the lavage device 30 is sized to be grasped and manipulated by surgical personnel during the surgical procedure. Alternatively, the body 32 of the lavage device 30 may be various sizes and shapes, depending upon the context of use. For example, the body 32 may be sized and shaped such that the lavage device 30 fits within the patient. Each of the irrigation system and the suction system includes a power source. The power source may be shared between the irrigation and suction systems, or each may include a dedicated power source. Accordingly, the body 32 of the lavage device 30 may also include one or more switches to actuate one or more of the irrigation system, the suction system, or even the UV light apparatus 20.

The irrigation system of the lavage device 30 is configured for moving a fluid to the patient. A variety of fluids may be used, including but not limited to saline and water. The fluid may also include an antiseptic or antifungal solution. The irrigation system includes a conduit 34 with an inlet for receiving the fluid from a reservoir or other fluid source and an outlet 35 through which the fluid is expelled to the patient. The length of the conduit 34 may vary depending upon the application. The irrigation system may also include a pump for moving the fluid through the conduit 34 to the patient.

The suction system removes fluid from the patient after it has been expelled by the irrigation system. The suction system includes a conduit 36 with an inlet 37 adapted to be positioned in proximity to the patient and configured for receiving the fluid from the patient. The length of the conduit 36 of the suction system may vary depending upon the application. A pump may also be operatively connected to the conduit 36 for moving the fluid.

In the embodiment shown in the FIGs., both conduits 34, 36 may extend outwardly of the body 32 and terminate in a distal nozzle portion 38 defining the outlet 35 of the irrigation system and the inlet 37 of the suction system. A variety of different nozzle configurations may be available to surgical personnel during the surgical procedure. A nozzle with the suitable distal ends of the conduits 34, 36 can be attached to the body 32 and used in a surgical procedure as necessary for the circumstance.

As noted above, it is understood that the UV light apparatus 20 as described herein may be used with a variety of different lavage devices. A suitable lavage device for use with UV light apparatus 20 is sold as the INTERPULSE and is available from Stryker Instruments.

In one embodiment, the UV light apparatus 20 is manufactured as a component of a lavage device 30. In this arrangement, the UV light apparatus 20 is accommodated within a monolithic body of the lavage device 30. The UV light apparatus 20 and the lavage device 30 may share a power source and circuitry.

In another embodiment, the UV light apparatus 20 may be a self-contained unit that can be selectively attached to the body 32 of the lavage device 30. In one embodiment, an attachment member 48 comprising a double-sided adhesive foam strip is used between the housing 22 of the UVC light apparatus 20 and the body 32 of the lavage device 30. The adhesive strip 48 allows the UV light apparatus 20 to be secured to a variety of surfaces without the need for customized attachment. In addition, the housing 22 of the UVC light apparatus 20 has both curved and flat surfaces to correspond to curved or flat attachment surfaces on the lavage device 30.

The UVC light apparatus 20 is positioned such that UV light emitted is directed to the area being treated by the lavage device 30. As shown in FIGS. 1-4, the UVC light apparatus 20 is attached to a top surface of the lavage device 30. More particularly, the light source 24 is positioned at the distal end of the wand 28 adjacent the nozzle 38 defining the outlet and inlet of the irrigation and suction systems, respectively. In other embodiments, the light source 24 may be positioned at different positions relative to the body 32 of the lavage device 30, and the UVC light apparatus 20 may be shaped or sized accordingly.

Other means for attaching the UVC light apparatus 20 to a lavage device 30, or any other medical or non-medical device or tool, are contemplated. Such means may provide for permanent or temporary attachment to the lavage device 30. In one embodiment, the attaching means may include tabs configured to engage with and connect with the lavage device 30, for example, in a groove on the lavage device. Flexible fingers can be configured to flex outward when the UVC lighting apparatus 20 is being attached to extend around the lavage device 30. The fingers seat on opposing exterior surfaces of the lavage device 30 and apply an inward compressive force to maintain attachment with the lavage device. In another embodiment, attaching means on the UVC lighting apparatus 20 includes a tab with a receptacle to receive a mechanical fastener. The fastener extends through the receptacle and into the lavage device 30 to maintain the attachment. Attachment of the UVC light apparatus 20 to the lavage device 30 may also accomplish an electrical connection between the one or more systems and the UVC light apparatus.

The UVC light apparatus 20 is configured to emit and direct UV light onto a surgical area. In one embodiment, the light source 24 emits UV-C light with sufficiently short wavelengths destructive to bacteria, viruses, and other micro-organisms. In some embodiments, the light source 24 emits UV light within a spectrum of between about 100-280 nm, and in a more specific range of between about 240-280 nm. In one specific embodiment, the UV light is at a wavelength of about 254 nm. The UV light is mutagenic to the bacteria, viruses, and other micro-organisms and breaks the molecular bonds within micro-organismal DNA. This bond breaking destroys the DNA, which either destroys the element, renders the element harmless, or prohibits the growth and reproduction.

To effectively kill bacteria, the UV lighting apparatus 20 must provide an adequate energy dosage at the appropriate wavelength. The minimum effective germicidal energy dose for UV light is 3 mJ/cm2. The wavelength range most effective in killing the relevant strains of bacteria is between 200 and 280 nm, with 265 nm being the optimum wavelength. Thus, a preferred LED emits light at a wavelength of 265 nm (±5 nm). This wavelength will effectively kill bacteria if the energy dosage is above 3 mJ/cm2.

The total UV dosage is a function of optical power, contact area, and time of exposure. A preferred exposure time can be up to 120 seconds or more, based on surgical observation and previous studies using UV light to kill bacteria. The remaining design parameters are adjusted by varying the distance to target area, viewing angle, and the number of LED's.

When the UV light apparatus 20 used with a lavage device 30, the lavage device 30 is operated to deliver a fluid through the outlet 35 of the conduit 34 of the irrigation system to a surgical site of the patient. The fluid is removed from the surgical site of the patient through the conduit 36 of the suction system. UV light is selectively emitted from the light source 24 to the surgical site while delivering and removing the fluid. This method combines tissue debridement with the antimicrobial effects of UVC light. In this application, the UV light apparatus 20 may be used for a variety of medical purposes, including but not limited to cleaning and rinsing wounds, and irrigating and washing an organ.

Although the UVC light apparatus has been shown and described in considerable detail with respect to only a few exemplary embodiments thereof, it should be understood by those skilled in the art that we do not intend to limit the apparatus to the embodiments since various modifications, omissions and additions may be made to the disclosed embodiments without materially departing from the novel teachings and advantages, particularly in light of the foregoing teachings. Accordingly, we intend to cover all such modifications, omission, additions and equivalents as may be included within the spirit and scope of the UVC light apparatus as described herein and defined by the following claims. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures.

What is claimed is:

1. A device for treating a patient, the device comprising:
a pulsatile lavage device comprising a first conduit through which fluid is introduced into the patient and a second conduit through which the fluid is removed from the patient;
a UV light source to emit UV light into the patient; and
a body that houses the lavage device and the UV light source.

2. The device of claim 1, wherein the body comprises a switch to activate the lavage device and the UV light source.

3. The device of claim 1, wherein the UV light is UV-C light within a spectrum of between about 240-280 nm.

4. A device for treating a patient, the device comprising:
a body;
an irrigation system attached to the body to move fluid into the patient and comprising a first conduit through which the fluid moves and with an outlet through which the fluid is expelled;
a suction system attached to the body and comprising a second conduit with an inlet through which the fluid is removed from the patient; and
a UV light source attached to the body and with an outlet through which UV light is emitted into the patient.

5. The device of claim 4, further comprising a switch at the body to activate the irrigation system, the suction system, and the light source.

6. The device of claim 4, wherein the UV light is within a spectrum of between about 100-280 nm.

7. The device of claim 4, wherein the UV light is is UV-C light within a spectrum of between about 240-280 nm.

8. The device of claim 4, further comprising an optical cable that extends between the UV light source and the outlet.

9. The device of claim 4, further comprising a battery positioned in the body and connected to the UV light source to provide power to the UV light source.

10. The device of claim 4, wherein the outlet of the UV light source is positioned at the outlet of the irrigation system and the inlet of the suction system.

11. A method of treating a patient comprising:
positioning a device at a surgical site within a patient;
delivering a fluid from the device to the surgical site within the patient;
removing from the surgical site the fluid through the device; and
while delivering and removing the fluid, emitting UV light from the device to the surgical site.

12. The method of claim 11, further comprising moving the device within the patient while delivering and removing the fluid and emitting the UV light.

13. The method of claim 11, further comprising activating a switch on the device and delivering and removing the fluid and emitting the UV light.
